(19) United States
(12) Patent Application Publication
(10) Pub. No.: US 2009/0209843 A1
(43) Pub. Date: Aug. 20, 2009

(54) METHOD TO ACQUIRE IMAGE DATA SETS WITH A MAGNETIC RESONANCE APPARATUS

(76) Inventor: Gudrun Graf, Shenzen (CN)

Correspondence Address: SCHIFF HARDIN, LLP;PATENT DEPARTMENT, 6600 SEARS TOWER, CHICAGO, IL, 60606-6473, US

(21) Appl. No.: 12/372,849

(22) Filed: Feb. 18, 2009

(30) Foreign Application Priority Data
Feb. 18, 2008 (DE) 10 2008 009 670.9

(51) Int. Cl. A61B 5/055

(52) U.S. Cl. 600/410

(57) ABSTRACT

In a method to acquire image data sets with a magnetic resonance apparatus in the examination of a patient, and to generate post-processed images, in which different measurement protocols to acquire various image data sets are set manually and the image data sets are acquired, and all measurement protocols are stored in an examination-specific or patient-specific measurement protocol file. The processing parameter sets for post-processing of at least one part of the image data sets are set manually and the post-processed images are generated, and all processing parameter sets are stored in the examination-specific or patient-specific measurement protocol file. The measurement protocols stored in the measurement protocol file are automatically loaded and executed to acquire comparable image data sets in the implementation of a follow-up examination of the same patient. The processing parameter sets stored in the measurement protocol file are automatically loaded, and the newly acquired image data sets are processed to generate comparable post-processed images.

### BACKGROUND OF THE INVENTION

1. Field of the Invention

The present invention concerns a method to acquire image data sets with a magnetic resonance apparatus in the framework of an examination of a patient, and to generate post-processed images therefrom.

2. Description of the Prior Art

An examination (scan) of a patient with a magnetic resonance apparatus, different image data sets are acquired that the physician either needs to have prepared for the scanning procedure or requires for an actual subsequent diagnosis. Examples of this are localizer exposures that serve to prepare for the subsequent actual measurement and the later 3D image data set acquisition itself, wherein various image data sets are acquired with different weightings or acquisition parameters.

In any case, in the examination it is initially necessary for the operator directed by a physician, to first manually set all measurement protocols that serve for acquisition of the different image data sets, i.e. to manually set the measurement parameters (settings) as he or she prefers the images to be generated for the later diagnostic evaluation.

After the end of the image data set acquisition, thus when all individual measurement protocols have been executed, the post-processing of the individual image data sets ensues in the process of which the operator/physician again first manually sets or defines the processing parameters with which a portion or all acquired image data sets are then post-processed in a suitable image data processing device. Post-processed images are thereby generated that are then output to the user on a monitor. These images can be significantly different images among those that can be generated from the corresponding 3D image data sets. Various post-processed images also can be generated from an image data set based on different processing parameter sets, which is well known.

The post-processed images allow a diagnostically relevant evaluation of the examination. If the same medical questions are to be reviewed or answered in the scope of a follow-up examination, however, it is necessary to acquire optimally comparable image data sets to generate optimally comparable post-processed images in order to have a meaningful comparison capability between a previous examination and a subsequent examination. Because the user must typically manually generate or define the measurement protocols as well as processing parameters before the respective examination or image post-processing. The user must initially consult the corresponding measurement protocols/processing parameter sets of the prior examination in order to set these again for the present examination, which is very complicated as well as being prone to error. It is known to store the measurement protocols that were incorporated or executed in the examination of a patient in an examination-specific or patient-specific measurement protocol file so that the user can electronically recall them in the event of a follow-up examination and can import these protocols for execution. Nevertheless, the difficulties described in the context of the image post-processing (which ensues manually, as before) still result, in which the user must, as before, manually establish and define the corresponding image processing parameters. This is sometimes very complicated since a number of individual parameters must be defined in order to obtain optimal, significant images.

### SUMMARY OF THE INVENTION

An object of the invention is to provide a method that enables comparable image data as well as post-processed images to be acquired in the implementation of a follow-up examination.

This object is achieved in accordance with the invention by a method to acquire image data sets with a magnetic resonance apparatus in the framework of an examination of a patient, and to generate post-processed images, which includes the following basic steps.

Different measurement protocols to acquire various image data sets are set manually and the image data sets are acquired, and all measurement protocols are stored in an examination-specific or patient-specific measurement protocol file.

The processing parameter sets for post-processing of at least one part of the image data sets are set manually and the post-processed images are generated, and all processing parameter sets are stored in the examination-specific or patient-specific measurement protocol file,

The measurement protocols stored in the measurement protocol file are automatically loaded and executed to acquire comparable image data sets in the implantation of a follow-up examination of the same patient.

The processing parameter sets stored in the measurement protocol file are automatically loaded, and the newly acquired image data sets are processed to generate comparable post-processed images.

According to the invention, an all-encompassing measurement protocol file is generated in the implementation of a first examination, in which file are stored containing both all manually defined and executed measurement protocols and all manually defined processing parameter sets in the framework of the first post-processing of the first acquired image data sets. This measurement protocol file is stored specific to the examination or patient, thus is uniquely associated with a specific examination with respect to a specific patient. It contains all relevant data or parameters that are absolutely necessary in order to produce a quasi-identical image data acquisition and a quasi-identical image data post-processing in the framework of a follow-up examination in order to arrive in this manner at the most broadly comparable data sets and the most broadly comparable post-processed images. The measurement protocols and the processing parameter sets are preferably automatically written to the corresponding measurement protocol file so that it is ensured that all protocols/processing parameter sets therein are also actually recorded without an action of the user being required. Naturally, the possibility exists for the user to delete from the measurement protocol file a measurement protocol or a processing parameter set that was possibly incorrectly selected or, respectively, defined or that yields no usable result.

In each case, in the implementation of a follow-up examination the user must merely select the examination-specific or patient-specific measurement protocol file. An automatic importation of the measurement protocols as well as an automatic execution of the same subsequently ensues in order to generate, in the follow-up examination, the corresponding image data sets of precisely that type just defined by the measurement protocols, as the corresponding image post-processing also subsequently, automatically ensues using the automatically loaded and then executed processing parameter sets.

An extremely effective and efficient tool that is the same time simple to operate is therefore available to the user, with which tool the user can effect the data acquisition as well as the post-processing significantly more efficiently, more quickly and (in particular with regard to the diagnostic question) significantly more precisely. It is ensured solely in this manner that a reliably comparable data acquisition as well as a reliably comparable data post-processing ensue, and the user obtains comparable post-processed images with which the user can posit a diagnosis enabling a well-substantiated conclusion, for example about the course of an illness etc.

Although it is sufficient to always use the original measurement protocols or processing parameter sets (thus those defined in the context of the initial examination) in the implementation of a follow-up examination, it is also advantageous to provide the user with the possibility of variation to allow new measurement protocols or processing parameter sets to be defined whenever this is appropriate for various reasons. For this purpose, according to the invention that new measurement protocols and/or processing parameter sets can be set manually by the user in the implementation of a follow-up examination, and new image data sets are correspondingly acquired and/or new images are correspondingly generated in the post-processing. The new measurement protocols and/or processing parameter sets are automatically stored in the examination-specific or patient-specific measurement protocol file. The user thus has the ability to be able to expand the measurement protocol file with new measurement protocols or processing parameter sets so that the system is not static but rather variable or expandable if this is necessary, for example for diagnostic or other reasons. These new measurement protocols or processing parameter sets are in turn automatically stored in the measurement protocol file; the expanded measurement protocol file in its entirety then forms the basis of a later follow-up examination.

In order to provide the user with a simple possibility of intervening, to insert possible new measurement protocols or, respectively, processing parameter sets into the measurement sequence (measurement queue) or the post-processing workflow it can also be provided that, before and/or after automatic execution of the measurement protocols and/or the processing parameter sets, a query is output to the user to implement additional image data set acquisitions or additional image data set post-processings by manual input of new measurement protocols or new processing parameter sets. The user is thus asked (for example before the beginning of the image data acquisition or at the end thereof and before the beginning of the automatic post-processing) whether additional measurement protocols should be generated and executed; the same correspondingly applies with regard to additional processing parameter sets. The user thus always has a possibility of engagement to define additional protocols/post-processing procedures.

An image post-processing system is typically structured hierarchically or in specific menu levels in terms of software; this means that different post-processing modalities or post-processing types are possible in defined display menu levels or working menu levels. According to the invention, it is now possible to have the post-processed images automatically stored and/or displayed in a display menu level defined by the image-specific processing parameter sets. This means that the system automatically associates the specific post-processed images with the respective display menu level or working menu level that is associated with this type of image post-processing. These display menu levels (often called “cards” or “windows”) can then be correspondingly selected by the user. The post-processed image is then displayed to him. Insofar as it is necessary, he can effect additional image-specific or, respectively, menu level-specific processings in the respective display or working menu level, thus can vary the displayed image again via corresponding working tools that are associated with this type of image post-processing etc. It is also possible to again store such variations in the measurement protocol file, possibly as a modification of the original processing parameter set that has been conducted to generate this (now modified) image.

As already described, the measurement protocol file is advantageously not a static file; rather it can be varied by the user, such as by the described possibility to expand additional measurement protocols or processing parameter sets, such as by erasing a measurement protocol, or by providing a processing parameter set according to the invention. Such variations are then no longer available for a follow-up examination, but can be inserted again without further measures by the described expansion possibility.

In principle, it is also appropriate (not least for safety reasons) to request from the user a release signal before implementing the automatic execution of the measurement protocols and/or the processing parameter sets, and to begin the operation only when this release signal has also been provided.

### BRIEF DESCRIPTION OF THE DRAWINGS

FIG. 1 schematically shows a magnetic resonance apparatus to implement the method.

FIG. 2 schematically illustrates an embodiment of the method according to the invention.

### DESCRIPTION OF THE PREFERRED EMBODIMENTS

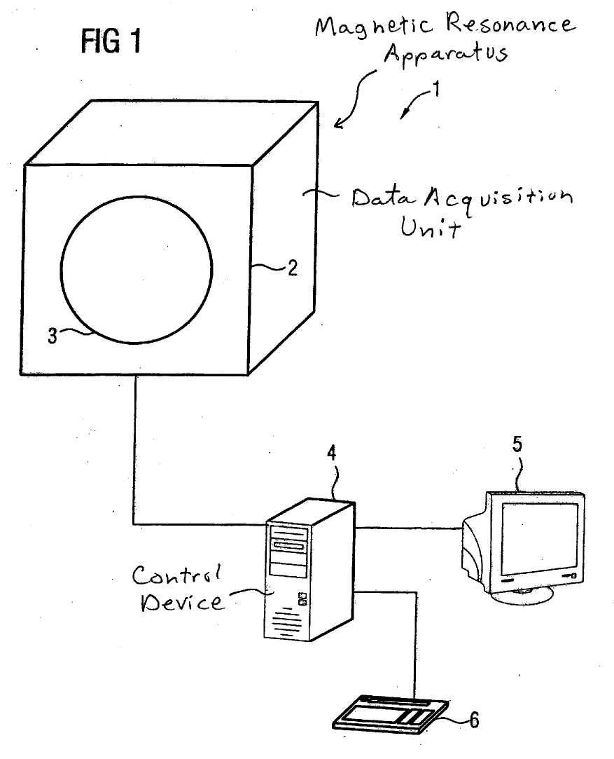

FIG. 1 shows a magnetic resonance apparatus 1 according to the invention (frequently also called a magnetic resonance system) having a data acquisition unit (scanner) with a patient receptacle 3 as well as the other components (basic field magnet, gradient coil, radio-frequency part etc.) sufficiently well known to those skilled in the art, which components are not shown in detail. A control device 4 that controls the operation both of the image data acquisition and the image post-processing is also provided. The generated images are displayed to the user on a monitor 5.

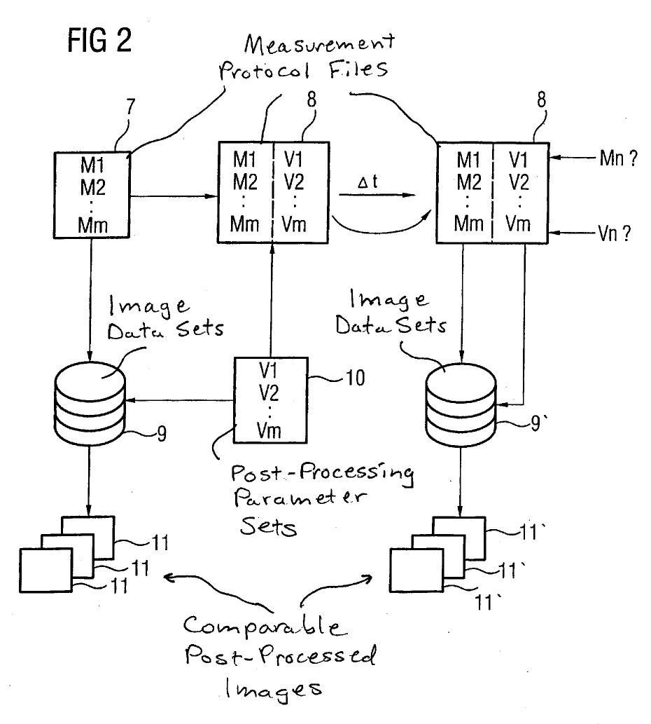

As can be seen from the basic diagram of FIG. 1, before beginning the first examination of a patient the user manually creates (with the assistance of an input unit 6 such as a keyboard, mouse, etc.) those measurement protocols that should form the basis of the subsequent image signal generation or, respectively, image data set generation. These are represented in FIG. 2 in the field marked with 7, the individual measurement protocols (of which there can be arbitrarily many), and the always-different measurements (labeled with M1, M2, . . . Mm).

After manual definition has occurred, the measurement protocols M1, . . . , Mm are automatically stored in a measurement protocol file 8, wherein this and all subsequently described procedures naturally ensue in corresponding working and storage means in the control device 4. All measurement protocols that are now subsequently executed are consequently archived in the measurement protocol file 8. This ensues specific to the examination or patient, meaning that a definite association with the examination or with the respective patient ensues so that the stored data can in every case be located again at a later point in time.

Starting from the measurement protocols, the measurement subsequently ensues, wherein different image data sets 9 are generated that are in turn likewise stored in the control device 4, specific to the examination or patient. An image post-processing now ensues in a suitable image processing module of the control device 4 to generate relevant images. For this purpose, the user again manually defines individual processing parameter sets via the input means 6 in the framework of the initial examination. These individual processing parameter sets are shown in Field 10 and marked with V1, V2, . . . , Vm. These can hereby be the most different processing parameter sets depending on what manner of post-processing it should be, for example whether it should be a “Maximum Intensity Projection” (MIP) or a “Volume Rendering,” etc.

After conclusion of the definition of the processing parameter sets, these are also automatically stored in the measurement protocol file 8 so that they are also archived and, together with the associated measurement protocols, are automatically associated with the examination or the patient. This measurement protocol file thus forms a parameter file that contains all relevant operating parameters for the image data acquisition as well as the image post-processing.

The image post-processing then ensues, meaning that the processing parameter sets V1, . . . , Vm are applied to the image data sets 9, either one part or all. In the end, multiple post-processed images 11 are obtained that are then output on the monitor 5, possibly structured according to corresponding hierarchy levels associated with the respective post-processing technique.

If, after an arbitrary amount of time has elapsed, the same patient should now be subjected to a follow-up examination in order to either check the treatment result or to monitor the possible progression of a potential illness etc., it is necessary to generate images comparable to a high degree. If the follow-up examination should be conducted after the elapsing of a specific amount of time (represented in FIG. 2 with Δt), for this purpose the measurement protocol file 8 of the first examination is selected after positioning the patient in the patient receptacle 3, wherein the measurement protocol file 8 was either still present in the control device 4 or was transferred to the control device 4 via data medium, intranet, internet etc. In each case, all information of the measurement protocol file 8 exists in the control device so that the user can access it. After selecting the measurement protocol file 8, all measurement protocols M1, . . . , Mm as well as all processing parameter sets V1, . . . , Vm stored therein are automatically imported and therefore automatically form the basis of the subsequent examination.

The automatic execution of the measurement protocols M1, . . . , Mm now ensues in the first step, possibly after preceding provision of a release signal by the user. Given the same positioning of the patient as in the initial examination, the same image data sets 9′ are acquired since the measurement protocols that form the basis of this image data set acquisition are the same as the measurement protocols that formed the basis of the acquisition of the image data sets 9 in the initial examination.

Before the automatic execution of the measurement protocols M1, . . . , Mm, or after this, a query is output to the user on the monitor 5 as to whether the user would like to define additional measurement protocols or possibly would like to delete a measurement protocol (then naturally before the execution). The integration of an additional measurement protocol is indicated with Mn in FIG. 2 as an example. In the event that this is the case, the user can again manually define and add this measurement protocol; it is then automatically stored in the measurement protocol file as a new measurement protocol Mn (possibly after execution has occurred) and is in turn available for a follow-up examination.

After acquisition of the image data sets 9′, the automates post-processing ensues using the processing parameters V1, . . . , Vm. This leads to the signal that superbly comparable images 11′ are obtained that are thus nearly identical to the images 11 in the framework of the initial examination; however, a possible pathological or diagnostically relevant chance can be detected in them. The images 11 and 11′ are therefore very comparable and diagnostically evaluable.

Before or after the processing of the image data sets 11′ with the processing parameter sets V1, . . . , Vm, the user is again asked whether an additional processing parameter set (represented with Vn in FIG. 2) should be added, or whether a different, already-present processing parameter set should possibly be erased. This means that an arbitrary expansion or deletion possibility also exists for the user within the processing parameter sets. Any change, be it an addition of a processing parameter set Vn or a deletion of an original processing parameter set, is in turn stored in the measurement protocol file 8. This (then modified) measurement protocol file 8 is available for a later follow-up examination in the framework of which new images comparable to a high degree with the images 11′ can again be acquired.

As already described, the most different processing techniques can be applied in the framework of the image post-processing. For example, “Maximum Intensity Projection” (MIP) is such a processing technique. In the framework of the method according to the invention, the parameter settings as they have been previously stored are automatically detected so that, in the images post-processed in the framework of the respective MIP method are comparable to a high degree with those of the preceding and subsequent examination. A 3D image data set is provided to the system. The MIP processing ensues automatically; the processing system automatically detects corresponding major vessels within the 3D image data set etc. and orients on previously selected settings, for example in that it rotates the 3D image data set until the vessels overlap as much as possible and it has found the underlying initial slice used for this. If image portions were truncated in the original MIP processing, these are also automatically detected and removed in the subsequent MIP processing.

If radial ranges were defined, these are also generated in the orientation selected in the previous post-processing. Multiple orientations can also be calculated, for example transversal source images, MIP ranges in a somersault processing and possible right-left rotations. The starting point can also be detected so that a right-left rotation is not swapped.

An addition technique is “Multi Planar Reformatting” (MPR). 3D measurements are frequently produced and 3D image data sets generated, however these are recalculated in other orientations. In that, due to the previous measurement, the image data processing system contains image data of the patient on which it can orient, these starting data can also be used as a basis for the recalculation. It is thereby ensured that the data of the pre-examination and post-examination can be post-processed as identically as possible, and thereby can be optimally compared. In the framework of what is known as neuro-3D processing, a number of patient-specific and measurement-specific settings are produced for what is known as “volume rendering”, and at the same time many settings for the functional imaging (fMRI) and difference data acquisition are also set. These data (which are likewise stored in the measurement protocol file) are retrieved from this, thus are also likewise present and can be used for the subsequent evaluation. A significantly better comparability of the data is hereby achieved, for example before and after operations. The data loaded directly into the menu levels (cards) associated with this neuro-3D processing enable the user to have volume rendering image presentation on the screen corresponding to the previous measurement as soon as the 3D image data measurement has ended. The functional images as well as the difference data are likewise generated and output as is defined in the initial examination.

The method for generation of comparable post-processed images according to the invention is also advantageous for perfusion measurements or spectroscopy.

By the definition of one or more new processing parameter sets in the follow-up examination, the possibility naturally also exists for the user to also post-process the image data sets acquired in the earlier examinations with these new processing parameter sets in order to also obtain corresponding, comparable images from the image data sets acquired earlier.

Although modifications and changes may be suggested by those skilled in the art, it is the intention of the inventor to embody within the patent warranted hereon all changes and modifications as reasonably and properly come within the scope of her contribution to the art.

I claim as my invention:

1. A method for generating and processing magnetic resonance images, comprising the steps of:
setting a measurement protocol, from among a plurality of different measurement protocols, to acquire a magnetic resonance image data set, and operating a magnetic resonance data acquisition device according to the selected measurement protocol to acquire an image data set of an examination subject in an examination;
storing the measurement protocol used to acquire the image data set in said examination in a measurement protocol file selected from the group consisting of an examination-specific measurement protocol file and a patient-specific measurement protocol file;
manually setting a processing parameter set for processing said image data acquired in said examination, to generate a processed image of the subject from said image data set;
storing the processing parameter set used to process the image data acquired in said examination in said measurement protocol file, linked to said image data set acquired in said examination that is stored in said measurement protocol file;
in a follow-up examination of said subject, subsequent to said examination, automatically loading the measurement protocol stored in said measurement protocol file that was used to acquire said image data set in said examination of said subject, and acquiring a follow-up image data set from the subject with said magnetic resonance data acquisition device; and
automatically loading the processing parameter set from said measurement protocol file that is linked to said image data set for said examination of said subject stored in said measurement protocol file, into a processor, and processing said follow-up image data in said processor according to said processing parameter set to obtain a follow-up processed image that is comparable to said processed image.

2. A method as claimed in claim 1 comprising, in said follow-up examination of said subject, manually setting a new measurement protocol, different from said measurement protocol used to acquire said image data set in said examination of said subject, and storing said new measurement protocol in said measurement protocol file linked to said processing parameter set that was previously stored in said measurement protocol file linked to said measurement protocol used to acquire said image data set in said examination of said subject.

3. A method as claimed in claim 1 comprising, contemporaneously with said follow-up examination of said subject, automatically displaying a query to an operator of said magnetic resonance data acquisition device as to whether, in said follow-up examination, a new measurement protocol should be set, different from said measurement protocol used to acquire said image data set from said subject in said examination.

4. A method as claimed in claim 1 comprising, in said follow-up examination, manually setting a new processing parameter set, different from said processing parameter set used to process the image data set acquired in said examination, and storing said new processing parameter set in said measurement protocol file linked with said image data set acquired in said measurement protocol used in said examination in said follow-up examination.

5. A method as claimed in claim 1 comprising, contemporaneously with said follow-up examination, displaying a query to an operator as to whether a new processing parameter set should be set for processing the follow-up image data set.

6. A method as claimed in claim 1 comprising automatically storing said processed image and said follow-up processed image according to a display menu level defined by an image-specific processing parameter set.

7. A method as claimed in claim 1 comprising allowing deletion of either a measurement protocol or a processing parameter set from said measurement protocol file by an action of an operator of said processor.

8. A method as claimed in claim 1 comprising requiring a manual release entry to be made into a controller of said magnetic resonance data acquisition device prior to operating said magnetic resonance data acquisition device to acquire image data from the subject using the measurement protocol that is automatically loaded from said measurement protocol file.

9. A method as claimed in claim 1 comprising requiring a manual release entry to be made into said processor before processing said follow-up image data set with the processing parameter set that is automatically loaded into said processor from said measurement protocol file.

10. A magnetic resonance system comprising:
a magnetic resonance data acquisition device;
a controller that operates said magnetic resonance data acquisition device;
a controller input connected to said controller allowing selection of a measurement protocol, from among a plurality of different measurement protocols, to acquire a magnetic resonance image data set, said controller being configured to operate said magnetic resonance data acquisition device according to the selected measurement protocol to acquire said image data set of an examination subject in an examination;
a memory in which the controller causes the measurement protocol used to acquire the image data set in said examination to be stored in a measurement protocol file selected from the group consisting of an examination-specific measurement protocol file and a patient-specific measurement protocol file;
an image processor;
an image processor input unit connected to said image processor allowing manual setting of a processing parameter set, said image processor being configured to process said image data acquired in said examination according to said processing, to generate a processed image of the subject from said image data set;
said image processor being configured to store the processing parameter set used to process the image data acquired in said examination in said measurement protocol file in said memory, linked to said image data set acquired in said examination that is stored in said measurement protocol file;
said controller being configured to, in a follow-up examination of said subject subsequent to said examination, automatically load the measurement protocol stored in said measurement protocol file in said memory that was used to acquire said image data set in said examination of said subject, and to operate magnetic resonance data acquisition device to acquire a follow-up image data set from the subject with said magnetic resonance data acquisition device; and
said image processor being configured to process said follow-up image data by automatically loading the processing parameter set from said measurement protocol file that is linked to said image data set for said examination of said subject stored in said measurement protocol file, and processing said follow-up image data in said processor according to said processing parameter set to obtain a follow-up processed image that is comparable to said processed image.
